(19) United States
(12) Patent Application Publication
(10) Pub. No.: US 2005/0019299 A1
(43) Pub. Date: Jan. 27, 2005

(54) Conditioning detergent compositions

(76) Inventors: Joseph J. LiBrizzi, Hillsborough, NJ (US); Irina Ganopolsky, Lawrenceville, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/623,999

(22) Filed: Jul. 21, 2003

(51) Int. Cl. A61K 7/06; A61K 7/11

(52) U.S. Cl. 424/70.17

(57) ABSTRACT

A conditioning detergent composition comprised of a surfactant portion comprising at least one of anionic, amphoteric, and nonionic surfactants and a conditioner portion comprising at least one branched cationic polymers and a silicone which is different from said branched cationic polymer and which is matrix soluble. The conditioning detergent composition imparts cleansing, wet detangling, dry detangling and manageability to hair and which is relatively non-irritating and thus suitable for use by young children and adults having sensitive skin and eyes. The conditioning detergent composition is able to provide such benefits in an aesthetically pleasing formulation without the need for suspending agents, opacifiers, or pearlizing agents.

### BACKGROUND OF THE INVENTION

1. Field of the Invention

This invention relates to conditioning detergent compositions suitable for use in personal cleansing application which not only impart cleansing, wet detangling, dry detangling and manageability properties to hair, but also which are relatively non-irritating and thus suitable for use by young children and adults having sensitive skin and eyes.

2. Description of the Prior Art

In the past, it has been considered desirable to cleanse hair and then to condition it after cleansing. For many years, it was necessary to perform these acts in two separate steps. However, with the advent of so-called “two-in-one” conditioning shampoos, it became possible to condition and cleanse simultaneously. Unfortunately, many of these two-in-one conditioning shampoos and body cleansers have proven to be relatively irritating to the eyes and skin and uncomfortable for use with children or sensitive adults. Therefore, it is an object of this invention to create a conditioning shampoo that has good cleansing ability, excellent conditioning properties and has a low degree of ocular and skin irritation.

One approach to providing hair conditioning benefits to a shampoo is described in U.S. Pat. No. 5,932,202, which discloses a composition comprised of an ethoxylated alkyl sulfate surfactant combined with an amphoteric surfactant; a cationic cellulosic polymer and a water insoluble non-volatile conditioning agent. While this shampoo claims to have optimized the conditioning properties of its cellulosic polymer by selection of a particular surfactant combination, that surfactant combination is not known as being gentle to the eyes and skin. Moreover, cationic cellulosic polymers are often disadvantageously associated with leaving an “unclean” residue to the hair and skin. Further, it is necessary to use a suspending agent for the insoluble conditioning agent in order to produce an aesthetically pleasing formulation. Typically, such formulations that required the use of a stabilizer are prone to separation and are capable of yielding only opaque products.

It would be desirable to have a conditioning composition that would not only impart cleansing, wet detangling, dry detangling and manageability properties to hair, but would also have a low degree of ocular and skin irritation. It would also be desirable to have such a conditioning detergent composition in a clear or translucent, aesthetically pleasing formulation without the need for adding pearlizers, opacifiers, and suspending agents thereto.

### SUMMARY OF THE INVENTION

In accordance with this invention, there is provided a conditioning detergent composition comprising:
a surfactant portion comprising at least one of the following:
   1. a nonionic surfactant;
   2. an amphoteric surfactant; and
   3. an anionic surfactant; and
a conditioner portion comprising:
   1. at least one branched cationic polymer; and
   2. at least one silicone that is different from the branched cationic polymer and is matrix soluble.

The composition of this invention, when used in a shampoo or body cleanser, possesses superior conditioning properties as well as one or more of the following properties: cleansing, shine, low dry static, softness, wet detangling, dry detangling, manageability, and low degree of ocular irritation. In addition, the composition may be made into various, aesthetically pleasing consumer cleansing products without the need for pearlizers, suspending agents or opacifiers.

While not bound by theory, it is believed that the combination of a branched cationic polymer and a matrix soluble silicone is an improvement over current systems containing macro emulsified silicone, such as, polydimethyl siloxane, methyl terminated (dimethicone) and polydimethyl siloxane, hydroxyl terminated (dimethiconol), due to its ability to deposit on hair or skin, yet still demonstrate ease of removal, and therefore reduced build-up, upon subsequent washings. Deposition of the micro emulsified silicon can occur via several mechanisms including electrostatic charge (if the silicone is cationic) or reduced solubility upon dilution resulting in a coating effect of insoluble material on the substrate. To enhance the deposition of silicone to hair and skin, a branched, cationic polymer is added.

It is believed that the branched, cationic polymer enhances deposition of the silicon via entrapment and, in some cases, association of the silicone. This effect could also occur with linear polymers, however, branched polymers are probably more effective due to their reduced packing density allowing for more physical room for entrapment and/or association. The cationic polymer entrapped or complexed silicone is then preferentially deposited to the negatively charged substrate (skin and hair) due to the attraction of the polymer cationic charge of the polymer. The reduced build-up on hair may be due to the fact that the silicone employed in this technology is soluble in the cleanser matrix (microemulsifiable). Micro emulsions, which because of their small droplet size (typically below 0.10 μm) cannot scatter light and therefore appear transparent, are formed spontaneously in conjunction with a surfactant. Any mechanical means used to mix the micro emulsion has no bearing on formation of the micro emulsion or particle size. In contrast, macroemulsions, which possess a droplet size typically greater than 0.15 μm, will scatter light due to their size and therefore appear opaque. Additionally, macroemulsions require both a surfactant to stabilize the emulsion droplets and mechanical energy to form droplets. Additional mechanical energy has the effect of lowering the droplet size. It is important to note that macroemulsions, unlike microemulsions, require a reasonable amount of mechanical energy to re-emulsify the deposited material from the substrate and wash it away. Microemulsions, on the other hand, require no mechanical energy to re-emulsify the deposited material and, therefore, can remove it easily from a substrate. During a wash cycle, the only means of mechanical action available would be the scrubbing of the hair or body with hand or bath implement. Upon repeated wash cycles that consist of removal of dirt and previously deposited material and deposition of new material, it is easy to see how a microemulsified material that is matrix soluble and requires no mechanical energy to re-emulsify might be easier to remove than a material that requires macroemulsification and the associated mechanical energy required for its removal.

### DETAILED DESCRIPTION OF PREFERRED EMBODIMENTS

It is believed that one skilled in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference.

By “matrix soluble” is meant capable of forming a microemulsion in the matrix, i.e, surfactant base, having a droplet radii below 0.15 μm.

The invention relates to a conditioning detergent composition comprising:
a surfactant portion comprising at least one of the following:
   1. a nonionic surfactant;
   2. an amphoteric surfactant; and
   3. an anionic surfactant; and
a conditioner portion comprising:
   1. at least one branched cationic polymer; and
   2. at least one silicone that is different from the branched cationic polymer and is matrix soluble.

The conditioning detergent composition is preferably comprised of, based upon the total weight of the conditioning detergent composition, from about 5 percent to about 20 percent, and more preferably from about 10 percent to about 15 percent of a surfactant portion and from about 0.1 percent to about 6 percent, preferably from about 0.5 percent to about 5 percent, and more preferably from about 1 percent to about 3 percent, of a conditioner portion.

The surfactant portion of the present invention contains nonionic, amphoteric and/or anionic surfactants. Preferably a mixture of nonionic, amphoteric and anionic surfactant is used and the weight ratio between the amphoteric surfactant and the anionic surfactant may range from about 3:1 to about 1:3, and preferably from about 2:1 to about 1:2. The weight ratio of the amphotericlanionic surfactant combination:non-ionic surfactant may vary widely, and preferably is about 2:1 to about 1:2. The nonionic surfactant is present in an amount, based upon the total weight of the conditioning detergent composition, of from about 0.1 percent to about 10 percent, preferably from about 1 percent to about 10 percent, and more preferably from about 4 percent to about 8 percent. The amphoteric surfactant is present in an amount, based upon the total weight of the conditioning detergent composition, of from about 0.5 percent to about 10 percent, preferably from about 1 percent to about 8 percent, and more preferably from about 2 percent to about 6 percent. The anionic surfactant is present in the conditioning detergent composition in an amount from about 1.0 percent to about 10 percent, preferably from about 1 percent to about 8 percent, and more preferably from about 1 percent to about 6 percent, based on the overall weight of the conditioning detergent composition.

One class of nonionic surfactants useful in the present invention are polyoxyethylene derivatives of polyol esters, wherein the polyoxyethylene derivative of polyol ester (1) is derived from (a) a fatty acid containing from about 8 to about 22, and preferably from about 10 to about 14 carbon atoms, and (b) a polyol selected from sorbitol, sorbitan, glucose, α-methyl glucoside, polyglucose having an average of about 1 to about 3 glucose residues per molecule, glycerin, pentaerythritol and mixtures thereof, (2) contains an average of from about 10 to about 120, and preferably about 20 to about 80 oxyethylene units; and (3) has an average of about 1 to about 3 fatty acid residues per mole of polyoxyethylene derivative of polyol ester.

Examples of preferred polyoxyethylene derivatives of polyol esters include, but are not limited to PEG-80 sorbitan laurate and Polysorbate 20. PEG-80 sorbitan laurate, which is a sorbitan monoester of lauric acid ethoxylated with an average of about 80 moles of ethylene oxide, is available commercially from ICI Surfactants of Wilmington, Del. under the tradename, “Atlas G4280.” Polysorbate 20, which is the laurate monoester of a mixture of sorbitol and sorbitol anhydrides condensed with approximately 20 moles of ethylene oxide, is available commercially from ICI Surfactants of Wilmington, Del. under the tradename “Tween 20.”

Another class of suitable nonionic surfactants includes long chain alkyl glucosides or polyglucosides, which are the condensation products of (a) a long chain alcohol containing from about 6 to about 22, and preferably from about 8 to about 14 carbon atoms, with (b) glucose or a glucose-containing polymer. The alkyl gluocosides have about 1 to about 6 glucose residues per molecule of alkyl glucoside. A preferred glucoside is decyl glucoside, which is the condensation product of decyl alcohol with a glucose polymer and is available commercially from Henkel Corporation of Hoboken, N.J. under the tradename, “Plantaren 2000.”

The compositions of the present invention may also contain an amphoteric surfactant. As used herein, the term “amphoteric” shall mean: 1) molecules that contain both acidic and basic sites such as, for example, an amino acid containing both amino (basic) and acid (e.g., carboxylic acid, acidic) functional groups; or 2) zwitterionic molecules which possess both positive and negative charges within the same molecule. The charges of the latter may be either dependent on or independent of the pH of the composition. Examples of zwitterionic materials include, but are not limited to, alkyl betaines and amidoalkyl betaines. The amphoteric surfactants are disclosed herein without a counter ion. One skilled in the art would readily recognize that under the pH conditions of the compositions of the present invention, the amphoteric surfactants are either electrically neutral by virtue of having balancing positive and negative charges, or they have counter ions such as alkali metal, alkaline earth, or ammonium counter ions.

Commercially available amphoteric surfactants are suitable for use in the present invention and include, but are not limited to amphocarboxylates, alkyl betaines, amidoalkyl betaines, amidoalkyl sultaines, amphophosphates, phosphobetaines, pyrophosphobetaines, carboxyalkyl alkyl polyamines and mixtures thereof.

Examples of suitable amphocarboxylate compounds include those of the formula:
A—CONH(CH₂)ₓN⁺R₅R₆ R⁷
wherein

A is an alkyl or alkenyl group having from about 7 to about 21, and preferably from about 10 to about 16 carbon atoms;
x is an integer of from about 2 to about 6;
R₅ is hydrogen or a carboxyalkyl group containing from about 2 to about 3 carbon atoms, and preferably is hydrogen;
R₆ is a hydroxyalkyl group containing from about 2 to about 3 carbon atoms or is a group of the formula:
   R₈—O—(CH₂)ₙCO₂
wherein
   R₈ is an alkylene group having from about 2 to about 3 carbon atoms and n is 1 or 2; and
R₇ is a carboxyalkyl group containing from about 2 to about 3 carbon atoms; Preferably, the amphocarboxylate compound is an imidazoline surfactant, and more preferably a disodium lauroamphodiacetate, which is commercially available from Mona Chemical Company of Paterson, N.J. under the tradename, “Monateric 949J.” When an amphocarboxylate is used in the conditioning detergent composition, it should be present in an amount of about 0.5 percent to about 10 percent, and preferably from about 0.5 percent to about 6 percent, based on the overall weight of the composition.

Examples of suitable alkyl betaines include those compounds of the formula:
B—N+R₉R₁₀(CH₂)ₚCO₂⁻
wherein
   B is an alkyl or alkenyl group having from about 8 to about 22, and preferably from about 8 to about 16 carbon atoms;
   R₉ and R₁₀ are each independently an alkyl or hydroxyalkyl group having from about 1 to about 4 carbon atoms; and
   p is 1 or 2.

A preferred betaine for use in the present invention is lauryl betaine, available commercially from Albright & Wilson, Ltd. of West Midlands, United Kingdom as “Empigen BB/J.” If present, the alkyl betaine should be used in an amount, based on the overall weight of the composition, of from about 0.25 percent to about 10 percent, preferably from about 0.25 percent to about 8 percent, and more preferably, from about 0.25 percent to about 5 percent.

Examples of suitable amidoalkyl betaines include those compounds of the formula:
D—CO—NH(CH₂)_{q}—N⁺R₁₁R₁₂(CH₂)ₘCO₂⁻
wherein
   D is an alkyl or alkenyl group having from about 7 to about 21, and preferably from about 7 to about 15 carbon atoms;
   R₁₁ and R₁₂ are each independently an alkyl or hydroxyalkyl group having from about 1 to about 4 carbon atoms;
   q is an integer from about 2 to about 6; and m is 1 or 2.
      A preferred amidoalkyl betaine is cocamidopropyl betaine, available commercially from Goldschmidt Chemical Corporation of Hopewell, Va. under the tradename, “Tegobetaine L7.” When present in the conditioning detergent compositions of this invention, the amidoalkyl betaine should be used in an amount of from about 0.25 percent to about 10 percent, preferably from about 0.25 percent to about 8 percent, and more preferably from about 0.25 percent to about 5 percent, based on the overall weight of the composition.

Examples of suitable amidoalkyl sultaines include those compounds of the formula 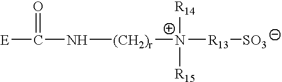
wherein
   E is an alkyl or alkenyl group having from about 7 to about 21, and preferably from about 7 to about 15 carbon atoms;
   R₁₄ and R₁₅ are each independently an alkyl, or hydroxyalkyl group having from about 1 to about 4 carbon atoms;
   r is an integer from about 2 to about 6; and
   R₁₃ is an alkylene or hydroxyalkylene group having from about 2 to about 3 carbon atoms;

Preferably the amidoalkyl sultaine is cocamidopropyl hydroxysultaine, available commercially from Rhone-Poulenc Inc. of Cranbury, N.J. under the tradename, “Mirataine CBS.” When present in the conditioning detergent compositions of this invention, it should be used in an amount of from about 0.5 percent to about 10 percent, preferably from about 1.0 percent to about 6 percent, and more preferably from about 1.5 percent to about 5 percent, based on the overall weight of the composition.

Examples of suitable amphophosphate compounds include those of the formula: 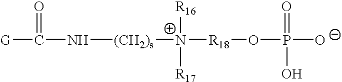
wherein
   G is an alkyl or alkenyl group having about 7 to about 21, and preferably from about 7 to about 15 carbon atoms;
      s is an integer from about 2 to about 6;
      R₁₆ is hydrogen or a carboxyalkyl group containing from about 2 to about 3 carbon atoms;
      R₁₇ is a hydroxyalkyl group containing from about 2 to about 3 carbon atoms or a group of the formula:
         R₁₉—O—(CH₂)ₜ—CO₂⁻
      wherein
         R₁₉ is an alkylene or hydroxyalkylene group having from about 2 to about 3 carbon atoms and t is 1 or 2; and
      R₁₈ is an alkylene or hydroxyalkylene group having from about 2 to about 3 carbon atoms.

Preferably the amphophosphate compounds are sodium lauroampho PG-acetate phosphate, available commercially from Mona Industries of Paterson, N.J. under the tradename, “Monateric 1023,” and those disclosed in U.S. Pat. No. 4,380,637, which is incorporated herein by reference, with sodium lauroampho PG-acetate phosphate being most preferred.

Examples of suitable phosphobetaines include those compounds of the formula: 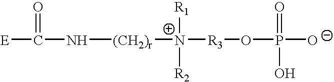
wherein E, r, R₁, R₂ and R₃, are as defined above. Preferably the phosphobetaine compounds are those disclosed in U.S. Pat. Nos. 4,215,064, 4,617,414, and 4,233,192.

Examples of suitable pyrophosphobetaines include those compounds of the formula: 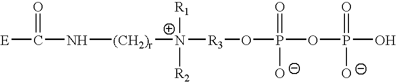
wherein E, r, R₁, R₂ and R₃, are as defined above. Preferably the pyrophosphobetaine compounds are those disclosed in U.S. Pat. Nos. 4,382,036, 4,372,869, and 4,617,414, which are all incorporated herein by reference.

Examples of suitable carboxyalkyl alkylpolyamines include those of the formula: 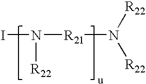
wherein
   I is an alkyl or alkenyl group containing from about 8 to about 22, and preferably from about 8 to about 16 carbon atoms;
   R₂₂ is a carboxyalkyl group having from about 2 to about 3 carbon atoms;
   R₂₁ is an alkylene group having from about 2 to about 3 carbon atoms and
   u is an integer from about 1 to about 4.

Preferably the carboxyalkyl alkyl polyamine is sodium carboxymethyl coco polypropylamine, available commercially from Akzo Nobel Surface Chemistry under the tradename, “Ampholak 7CX/C.” When present in the conditioning detergent compositions of this invention, it should be used in an amount of from about 0.5 percent to about 10 percent, preferably from about 1.0 percent to about 8 percent, and more preferably from about 2.0 percent to about 6.0 percent, based on the overall weight of the composition.

In a preferred embodiment, the amphoteric surfactant portion of the conditioning detergent composition is comprised of a mixture of amphoteric surfactants, such as amphocarboxylate and alkyl betaine, or amphocarboxylate and amidoalkyl betaine. In this embodiment, the amphocarboxylate is present in the conditioning detergent composition in an amount, based upon the total weight of the conditioning detergent composition, of from about 0.5 percent to about 9.5 percent and the alkyl betaine or amidoalkyl betaine is present in an amount, based upon the total weight of the conditioning detergent composition, of from about 9.5 percent to about 0.5 percent.

The conditioning detergent compositions of this embodiment may also contain at least one anionic surfactant. Preferably, the anionic surfactant is selected from the following classes of surfactants: 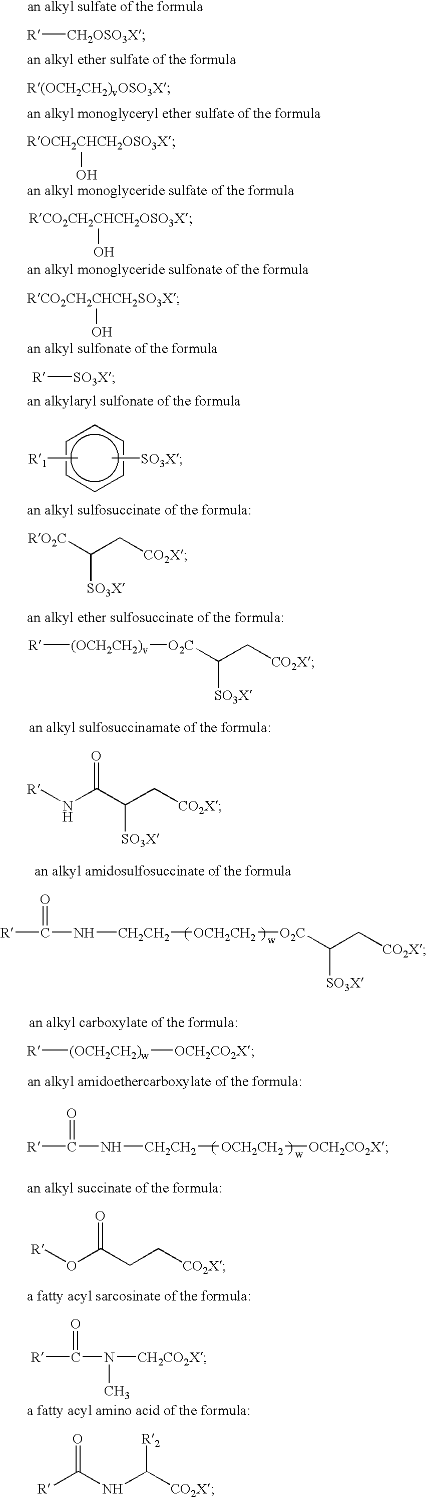 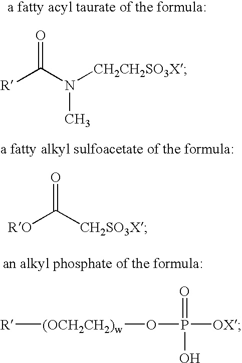
wherein
   R′ is an alkyl group having from about 7 to about 22, and preferably from about 7 to about 16 carbon atoms,
R′₁ is an alkyl group having from about 1 to about 18, and preferably from about 8 to about 14 carbon atoms,
R′₂ is a substituent of a natural or synthetic α-amino acid,
X′ is selected from the group consisting of alkali metal ions, alkaline earth metal ions, ammonium ions, and ammonium ions substituted with from about 1 to about 3 substituents, each of the substituents may be the same or different and are selected from the group consisting of alkyl groups having from 1 to 4 carbon atoms and hydroxyalkyl groups having from about 2 to about 4 carbon atoms and
v is an integer from 1 to 6;
w is an integer from 0 to 20;
   and mixtures thereof. Preferably the anionic surfactant is comprised of sodium trideceth sulfate, sodium laureth sulfate, disodium laureth sulfosuccinate, or mixtures thereof. Sodium trideceth sulfate is the sodium salt of sulfated ethoxylated tridecyl alcohol that conforms generally to the following formula, C₁₃H₂₇(OCH₂CH₂)ᵣOSO₃Na, where n has a value between 1 and 4, and is commercially available from Stepan Company of Northfield, Ill. under the tradename, “Cedapal TD-403M.” Sodium laureth sulfate is available from Albright & Wilson, Ltd. West Midlands, United Kingdom under the tradename, “Empicol 0251/70-J.” Disodium laureth sulfosuccinate is available commercially from Albright & Wilson, Ltd. of West Midlands, United Kingdom under the tradename, “Empicol SDD.”

In a preferred embodiment, the conditioning detergent compositions of the present invention contain a surfactant portion comprised of, based upon the total weight of the conditioning detergent composition, from about 1 percent to about 5 percent sodium trideceth sulfate; from about 2 percent to about 6 percent cocamidopropyl betaine; from about 0.5 percent to about 2.0 percent lauroamphodiacetate; and from about 3 percent to about 8 percent of PEG 80 sorbitan laurate.

The conditioner portion of the present invention preferably comprises:
1. at least one branched cationic polymer; and
2. at least one silicone that is different from the branched cationic polymer and is matrix soluble.

The amount of branched cationic polymer may range, based upon the total weight of the conditioning detergent composition, from about 0.001 percent to about 5.0 percent, preferably from about 0.01 percent to about 3.0 percent, and more preferably from about 0.1 to about 1.5 percent. The amount of matrix soluble silicone may range, based upon the total weight of the conditioning detergent composition, from about 0.01 percent to about 8.0 percent, preferably from about 0.1 percent to about 5.0 percent, and more preferably from about 0.5 to about 5.0 percent.

The amount of branched cationic polymer conditioner component may range, based upon the total weight of the conditioning portion of the conditioning detergent composition, from about 6 percent to about 70 percent, preferably from about 10 percent to about 60 percent, and more preferably from about 15 to about 55 percent.

In one embodiment, the conditioning portion contains a branched cationic polymer: silicone conditioner combination in a weight ratio of from about 0.5:1 to about 1:5.

Examples of suitable cationic branched polymers include polymers or copolymers of the following:
(a) ethylenically unsaturated monomers;
(b) silicones;
(c) polysaccharides; and
(d) vinylpyrrolidone monomers

The cationic branched polymer is preferably a quaternary polymer.

Examples of suitable polymers and copolymers of ethylenically unsaturated monomers include the copolymer of acrylamidopropyltrimonium chloride and acrylamide sold under the trade name SALCARE® SC60 by CIBA.

Examples of suitable polymers and copolymers of silicones include but are not limited to cetyl triethylmonium dimethicone copolyol succinate sold under the tradename BIOSIL BASICS CETYLSIL S by BIOSIL Technologies, Inc. and steardimonium hydroxypropyl Panthenyl PEG-7 dimethicone phosphate chloride sold under the tradename PECOSIL PAN418 by Phoenix Chemical, Inc.

Examples of suitable cationic branched polymers of polysaccharides include guar hydroxypropyl trimonium chloride sold under the trade name JAGUAR C-17 by Rhodia, Inc.

Examples of suitable polymers and copolymers of vinylpyrrolidone include, but are not limited to vinylpyrrolidone/vinylimidazolium copolymers. Preferred vinylpyrrolidone/vinylimidazolium copolymers include the materials known as Polyquaternium-44, which is a vinylpyrrolidone/vinylimidazolium methosulfate copolymer that is commercially available from BASF Corporation under the tradename, “Luviquat Care” and Polyquaternium 16, which is a vinylpyrrolidone/vinylimidazolium methyl chloride copolymer that is commercially available from BASF Corporation under the tradename, “Luviquat FC905;” and mixtures thereof.

Examples of suitable matrix soluble silicones include volatile silicones, non-volatile silicones, and mixtures thereof, with the non-volatile silicones being preferred. Examples of suitable water insoluble silicones include, for e.g., those set forth in U.S. Pat. No.: 5,932,202, the disclosure of which is hereby incorporated by reference.

Preferred volatile silicone conditioning agents have an atmospheric pressure boiling point less than about 220° C. Examples of suitable volatile silicones nonexclusively include trimethylsilylamodimethicone, phenyl trimethicone, polydimethylsiloxane having a viscosity less than about 5 cSt, polydimethylcyclosiloxanes, hexamethyldisiloxane, cyclomethicone fluids such as such as those available commercially from Dow Corning Corporation of Midland, Mich. under the tradename, “DC-345” and mixtures thereof.

Examples of suitable nonvolatile silicone conditioning agents nonexclusively include organo-substituted polysiloxanes, which are either linear or cyclic polymers of monomeric silicone/oxygen monomers and which nonexclusively include cetyl dimethicone; cetyl triethylammonium dimethicone copolyol phthalate; dimethicone copolyol; dimethicone copolyol lactate; hydrolyzed soy protein/dimethicone copolyol acetate; silicone quaternium 13; stearalkonium dimethicone copolyol phthalate; stearamidopropyl dimethicone and mixtures thereof; polyaryl siloxanes such as phenyl trimethicone; polyalkyl siloxanes such as the amino substituted amodimethicones; polyalkylarylsiloxanes; and derivatives there of and mixtures thereof.

Particularly suitable matrix soluble silicones for use in the invention include trimethylsilylamodimethicone, dimethicone copolyol, and amodimethicone, such as, DOW Corning 2-8566 Aminio Fluid available from Dow Corning Corporation and mixtures thereof.

Preferably, the conditioning detergent composition of the present invention is visually clear and is free of or substantially free of pearlizing agents, opacifiers, or suspending agents. By “substantially free of,” it is meant that the conditioning detergent compositions contains, based upon the total weight of the conditioning detergent composition, no more than 1.0 percent, preferably no more than 0.5 percent, and more preferably no more than 0.1 percent of a pearlizing agent, opacifier, or suspending agent. We have unexpectedly found that the conditioning detergent composition of the present invention results in an aesthetically pleasing formulation that possesses improved detangling and conditioning properties in the absence of pearlizing agents, opacifiers, and suspending agents. Without wishing to be bound by theory, we believe that the resulting formulations do not phase-separate as a result of the interaction between the surfactants and the silicones with the unique structure of the branched quaternary cationic polymer. By not having to use such stabilizing agents, et al., the conditioning detergent composition of the present invention may be colorless, clear, or translucent.

In embodiments wherein an opaque composition may be desired, the composition of the present invention may also include one or more optional ingredients nonexclusively including a pearlescent or opacifying agent, or a thickening agent. Other optional ingredients include secondary conditioners, humectants, chelating agents, and additives which enhance their appearance, feel and fragrance, such as colorants, fragrances, preservatives, pH adjusting agents, and the like. The pH of the conditioning detergent compositions of this invention is preferably maintained in the range of from about 5 to about 7.5, and more preferably from about 5.5 to about 7.2.

Commercially available pearlescent or opacifying agents which are capable of suspending water insoluble additives such as silicones and/or which tend to indicate to consumers that the resultant product is a conditioning shampoo are suitable for use in this invention. The pearlescent or opacifying agent is present in an amount, based upon the total weight of the composition, of from about 0 percent to about 3 percent, preferably from about 0.25 percent to about 2.5 percent, and more preferably, from about 0.5 percent to about 1.5 percent. Examples of suitable pearlescent or opacifying agents include, but are not limited to mono or diesters of (a) fatty acids having from about 16 to about 22 carbon atoms and (b) either ethylene or propylene glycol; mono or diesters of (a) fatty acids having from about 16 to about 22 carbon atoms (b) a polyalkylene glycol of the formula
HO—(JO)ₐ—H
wherein
   J is an alkylene group having from about 2 to about 3 carbon atoms; and a is 2 or 3; fatty alcohols containing from about 16 to about 22 carbon atoms; fatty esters of the formula
      KCOOCH₂L
wherein K and L independently contain from about 15 to about 21 carbon atoms;
   inorganic solids insoluble in the conditioning detergent composition, and mixtures thereof.

In a preferred embodiment, the pearlescent or opacifying agent is introduced to the conditioning detergent composition as a pre-formed, stabilized aqueous dispersion, such as that commercially available from Henkel Corporation of Hoboken, N.J. under the tradename, “Euperlan PK-3000.” This material is a combination of glycol distearate (the diester of ethylene glycol and stearic acid), Laureth-4 (CH₃(CH₂)₁₀CH₂(OCH₂CH₂)₄OH) and cocamidopropyl betaine and preferably is in a weight percent ratio of from about 25 to about 30: about 3 to about 15: about 20 to about 25, respectively.

Commercially available thickening agents which are capable of imparting the appropriate viscosity to the conditioning detergent compositions are suitable for use in this invention. If used, the thickener should be present in the conditioning detergent compositions in an amount sufficient to raise the Brookfield viscosity of the composition to a value of between about 500 to about 10,000 centipoise. Examples of suitable thickening agents nonexclusively include: mono or diesters of 1) polyethylene glycol of formula
HO—(CH₂CH₂O)_{z}H
wherein z is an integer from about 3 to about 200;
   and 2) fatty acids containing from about 16 to about 22 carbon atoms; fatty acid esters of ethoxylated polyols; ethoxylated derivatives of mono and diesters of fatty acids and glycerine; hydroxyalkyl cellulose; alkyl cellulose; hydroxyalkyl alkyl cellulose; and mixtures thereof. Preferred thickeners include polyethylene glycol ester, and more preferably PEG-150 distearate which is available from the Stepan Company of Northfield, Ill. or from Comiel, S.p.A. of Bologna, Italy under the tradename, “PEG 6000 DS”.

From about greater than 0 percent to about 4 percent, e.g. about 2 percent to about 3 percent, based upon the total weight of the conditioning detergent composition, of commercially available secondary conditioners may optionally be added into the conditioning detergent composition. These secondary conditioners may be comprised of from cationic cellulose derivatives; cationic guar derivatives; and a homopolymers or copolymers of a cationic monomer selected from:
a. a monomer having formula 1. 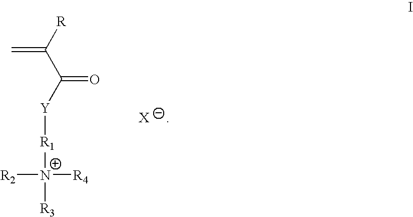
wherein
   R is H or CH₃,
   Y is O or NH,
   R₁ is an alkylene group having from about 2 to about 6, and preferably from about 2 to about 3 carbon atoms,
   R₂, R₃ and R₄ are each independently an alkyl group having from about 1 to about 22, and preferably from about 1 to about 4 carbon atoms, and
   X is a monovalent anion selected from halide and alkyl sulfate, or
b. diallyldimethylammonium chloride.

Examples of cationic cellulose derivatives include polymeric quaternary ammonium salts derived from the reaction of hydroxyethyl cellulose with a trimethylammonium substituted epoxide. The material known as Polyquaternium-10, commercially available from Amerchol Corporation of Edison, N.J. as “Polymer JR400,” is especially useful in this regard.

Another example of suitable secondary conditioners includes those compounds derived from acrylamidopropyl trimonium chloride which has the formula: 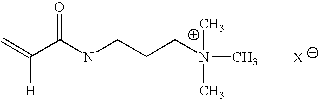
and more preferably is the copolymer of this monomer with acrylamide, the latter of which is available commercially from Allied Colloids, of Suffolk, Va. under the tradename, “Salcare SC60.”

Other preferred secondary conditioners are the cationic conditioning polymers that are derived from the monomer diallyidimethylammonium chloride. The homopolymer of this monomer is Polyquaternium-6, which is available commercially from Ciba Geigy Corporation under the tradename, “Salcare SC30.” The copolymer of diallyldimethylammonium chloride with acrylamide is known as Polyquaternium-7, and is also available from Ciba Geigy under the tradename “Salcare SC10.”

Commercially available humectants, which are capable of providing moisturization and conditioning properties to the conditioning detergent composition, are suitable for use in the present invention. The humectant is present in an amount of from about 0 percent to about 10 percent, preferably from about 0.5 percent to about 5 percent, and more preferably from about 0.5 percent to about 3 percent, based on the overall weight of the conditioning detergent composition. Examples of suitable humectants nonexclusively include: 1) water soluble liquid polyols selected from the group comprising glycerin, propylene glycol, hexylene glycol, butylene glycol, dipropylene glycol, and mixtures thereof; 2) polyalkylene glycol of the formula
HO—(R″O)_{b}—H
wherein R″ is an alkylene group having from about 2 to about 3 carbon atoms and b is an integer of from about 2 to about 10;
3) polyethylene glycol ether of methyl glucose of formula
   CH₃—C₆H₁₀O₅—(OCH₂CH₂)_{c}—OH
wherein c is an integer from about 5 to about 25;
4) urea; and 5) mixtures thereof, with glycerine being the preferred humectant.

Examples of suitable chelating agents include those which are capable of protecting and preserving the compositions of this invention. Preferably, the chelating agent is EDTA, and more preferably is tetrasodium EDTA available commercially from Dow Chemical Company of Midland, Mich. under the tradename, “Versene 100XL” and is present in an amount, based upon the total weight of the composition, from about 0 to about 0.5 percent, and preferably from about 0.05 percent to about 0.25 percent. Suitable preservatives include Quaternium-15, available commercially as “Dowicil 200” from the Dow Chemical Corporation of Midland, Mich., and are present in the composition in an amount, based upon the total weight of the composition, from about 0 to about 0.2 percent, and preferably from about 0.05 percent to about 0.10 percent.

The above described conditioning detergent composition may be prepared by combining the desired components in a suitable container and mixing them under ambient conditions in any conventional mixing means well known in the art, such as a mechanically stirred propeller, paddle, and the like. Although the order of mixing is not critical, it is preferable to pre-blend certain components, such as the fragrance and the nonionic surfactant before adding such components into the main mixture.

The conditioning detergent composition of the present invention is preferably used in personal cleansing applications nonexclusively including shampoos, gels such as shower gels, baths such as baby baths, washes such as body washes, and the like.

The invention illustratively disclosed herein suitably may be practiced in the absence of any component, ingredient, or step which is not specifically disclosed herein. Several examples are set forth below to further illustrate the nature of the invention and the manner of carrying it out. However, the invention should not be considered as being limited to the details thereof.

### EXAMPLES

All amounts of materials are given in parts by weight based on 100 parts of the overall formulation, unless stated otherwise.

### Example 1

### Preparation of Conditioning Detergent Composition

The amounts of the ingredients used to make the composition of Example 1 are shown in Table 1 below.

TABLE 1

| | | | | % |
|---|---|---|---|---|
| Tradename | INCI Name | Supplier | % Active | (wt/wt) |
| Monateric-949J | Disodium | Uniqema | 30 | 2.85 |
| | Lauroamphodiacetate | | | |
| Cedepal TD 403 | Sodium Trideceth (3) | Stepan Company | 30 | 9.50 |
| | Sulfate | | | |
| Carbopol AQUA SF1 | Carbomer | Noveon, Inc. | 30 | 5.00 |
| KESSCO PEG 6000 DS | PEG-150 Distearate | Stepan Company | 100 | 0.70 |
| Atlas G-4280 | POE 80 Sorbitan | Uniqema | 72 | 6.50 |
| | Monolaurate | | | |
| Jaguar C17 | Guar Hydroxypropyl | Rhodia, Inc. | 100 | 0.1 |
| | trimonium Chloride | | | |
| Tegobetaine L7 | Cocamidopropyl Betaine | Degussa | 30 | 13.3 |
| Polyox WSR 205 | PEG-14M | Dow Chemical | 100 | 0.05 |
| Glycerin, USP | Glycerin | Cognis | 100 | 1.9 |
| Pecosil PAN-418 | Steardimonium | Phoenix Chemical, | 37 | 1.0 |
| | Hydroxypropyl Panthenyl | Inc. | | |
| | PEG-7 Dimethicone | | | |
| | Phosphate Chloride | | | |
| Dow Corning 2-8566 Amino | Amodimethicone | Dow Corning | 100 | 1.0 |
| Fluid | | Corporation | | |
| Euperlan PK-3000 | Glycol Distearate & Laureth- | Cognis | 40 | 2.00 |
| | 4 & Cocamidopropyl Betaine | | | |
| Versene 100XL | Tetrasodium EDTA | Dow Chemical | 38 | 0.25 |
| Sodium Hydroxide, USP | Sodium Hydroxide, USP | | 100 | As needed |
| Deionized Water | Water | | 0 | QS to 100 |

Preparation of Pre-Mix:

Component amounts in this procedure were given in terms of parts by weight to prepare 100 parts of the pre-mix.

PreMix 1:

11 parts of PEG-150 Distearate were added to 89 parts of POE-80 Sorbitan Laurate in a Pyrex glass beaker with mixing. The blend was heated to 50C and mixing continued until the resulting pre-mixture was substantially clear and free of particulate.

PreMix 2:

2.5 parts of PEG-14M were added with 5.0 parts of Guar Hydroxypropyltrimonium Chloride to 92.5 parts Glycerin and mixed at room temperature until a homogenous suspension was formed.

Preparation of Main Mixture:

To 50.0 parts of Deionized water, 5.0 parts of Carbomer was added with agitation. 9.5 parts of Sodium Trideceth(3) Sulfate was added to the batch under agitation. 6.20 parts of PreMix 1 (PEG-150 Distearate/POE-80 Sorbitan Laurate blend) made as described above were added. While continuing mixing, 13.3 parts Cocamidopropyl Betaine followed by 2.85 parts Disodium Lauroamphodiacetate were added to the batch with continued mixing. 2.05 parts of PreMix 2 (PEG-14M/Guar Hydroxypropyltrimonium Chloride/Glycerin blend) made as described above was then added. While mixing continued, 1.0 parts of amodimethicone was added. The batch was mixed for at least one minute or until solution was homogenous. 1.0 parts of Steardimonium Hydroxypropyl Panthenyl PEG-7 Dimethicone Phosphate Chloride was added and mixed for at least one minute or until the solution was homogenous. Once mixture was homogenous, 0.05 parts Quaternium 15, 0.25 parts Tetrasodium EDTA and 2.0 Parts Euperlan PK3000 were added. The pH was adjusted to 6.5-7.0 with Sodium Hydroxide. QS to 100 parts with Deionized water.

### Example 2

### Preparation of Clear Conditioning Detergent Composition

The amounts of the ingredients for the composition of Example 2 are shown in Table 2 below.

TABLE 2

| | | | | % |
|---|---|---|---|---|
| Tradename | INCI Name | Supplier | % Active | (wt/wt) |
| Monateric-949J | Disodium | Uniqema | 30 | 2.85 |
| | Lauroamphodiacetate | | | |
| Cedepal TD 403 | Sodium Trideceth (3) | Stepan Company | 30 | 9.50 |
| | Sulfate | | | |
| Carbopol AQUA SF1 | Carbomer | Noveon, Inc. | 30 | 5.00 |
| Jaguar C17 | Guar Hydroxypropyl | Rhodia, Inc. | 100 | 0.1 |
| | trimonium Chloride | | | |
| Tegobetaine L7 | Cocamidopropyl Betaine | Degussa | 30 | 13.3 |
| Polyox WSR 205 | PEG-14M | Dow Chemical | 100 | 0.05 |
| Glycerin, USP | Glycerin | Cognis | 100 | 1.9 |
| Pecosil PAN-418 | Steardimonium | Phoenix Chemical, | 37 | 1.0 |
| | Hydroxypropyl Panthenyl | Inc. | | |
| | PEG-7 Dimethicone | | | |
| | Phosphate Chloride | | | |
| Dow Corning 2-8566 Amino | Amodimethicone | Dow Corning | 100 | 1.0 |
| Fluid | | Corporation | | |
| Versene 100XL | Tetrasodium EDTA | Dow Chemical | 38 | 0.25 |
| Sodium Hydroxide, USP | Sodium Hydroxide, USP | | 100 | As needed |
| Deionized Water | Water | | 0 | QS to 100 |

Preparation of Pre-Mix:

Component amounts in this procedure were given in terms of parts by weight to prepare 100 parts of the pre-mix.

PreMix 1:

To 92.5 parts Glycerin add 2.5 parts of PEG-14M and 5.0 parts of Guar Hydroxypropyltrimonium Chloride and mix at room temperature until a homogenous suspension is formed.

Preparation of Main Mixture:

To 50.0 parts of Deionized water add 5.0 parts of Carbomer with agitation. Add 9.5 parts of Sodium Trideceth(3) Sulfate to the batch under agitation. While continuing mixing, add to the batch add 13.3 parts Cocamidopropyl Betaine followed by 2.85 parts Disodium Lauroamphodiacetate. Add 2.05 parts of PreMix 1 (PEG-14M/Guar Hydroxypropyltrimonium Chloride/Glycerin blend) made as described above. While continuing mixing, add 1.0 parts of amodimethicone. Mix for at least one minute or until solution is homogenous. Add 1.0 parts of Steardimonium Hydroxypropyl Panthenyl PEG-7 Dimethicone Phosphate Chloride and mix for at least one minute or until solution is homogenous. Once mixture is homogenous, add 0.05 parts Quaternium 15, 0.25 parts Tetrasodium EDTA and 2.0 Parts Euperlan PK3000. Adjust pH to 6.5-7.0 with Sodium Hydroxide. QS to 100 parts with Deionized water. A visually clear conditioning detergent composition results.

1. A conditioning detergent composition comprising:
a surfactant portion comprising at least one of the following:
1. a nonionic surfactant;
2. an amphoteric surfactant; and
3. an anionic surfactant; and
a conditioner portion comprising:
1. at least one cationic branched polymer; and
2. at least one silicone which is different from said cationic branched polymer and is matrix soluble.

2. The detergent composition of claim 1 which is visually clear.

3. The detergent composition of claim 2 which is substantially free of pearlizing agents, opacifiers and suspending agents.

4. The detergent composition of claim 1 comprising, based upon the total weight of the conditioning detergent composition, from about 5 percent to about 20 percent of the surfactant portion and from about 0.1 percent to about 6.0 percent of the conditioning portion.

5. The detergent composition of claim 1 comprising, based upon the total weight of the conditioning detergent composition, from about 10 percent to about 15 percent of the surfactant portion and from about 0.5 percent to about 5.0 percent of the conditioning portion.

6. The detergent composition of claim 1, wherein the conditioner portion is present in an amount, based upon the total weight of the detergent composition, from about 0.1 percent to about 1.5 percent.

7. The detergent composition of claim 1, comprising based upon the total weight of the conditioning detergent composition, from about 0.001 to about 5.0 percent of the cationic branched polymer.

8. The detergent composition of claim 1, comprising based upon the total weight of the conditioning detergent composition, from about 0.01 to 3.0 percent of the cationic branched polymer.

9. The detergent composition of claim 1, comprising based upon the total weight of the conditioning detergent composition, from about 0.1 to about 1.5 percent of the cationic branched polymer.

10. The detergent composition of claim 1, comprising based upon the total weight of the conditioning detergent composition, from about 0.01 to about 8 percent of the matrix soluble silicone.

11. The detergent composition of claim 1, comprising based upon the total weight of the conditioning detergent composition, from about 0.1 to about 5 percent of the matrix soluble silicone.

12. The detergent composition of claim 1, comprising based upon the total weight of the conditioning detergent composition, from about 0.5 to about 5 percent of the matrix soluble silicone.

13. The detergent composition of claim 1 wherein said at least one cationic branched polymer comprises at least one polymer or copolymer of at least one of the following:
(a) an ethylenically unsaturated monomer;
(b) a silicone;
(c) a polysaccharide; and
(d) a vinylpyrrolidone monomer.

14. The detergent composition of claim 13, wherein said cationic branched polymer comprises an ethylenically unsaturated monomer which is a copolymer of acrylamidopropyltrimonium chloride and acrylamide.

15. The detergent composition of claim 13, wherein said cationic branched polymer comprises a silicone which is selected from cetyl triethylmonium dimethicone copolyol succinate, steardimonium hydroxypropyl panthenyl PEG-7 Dimethicone Phosphate Chloride and mixtures thereof.

16. The detergent composition of claim 13, wherein said cationic branched polymer comprises a polysaccharide which is guar hydroxypropyl trimonium chloride.

17. The detergent composition of claim 13, wherein said cationic branched polymer comprises a vinylpyrrolidone monomer which is selected from a copolymer of vinylpyrrolidone/vinylimidazolium methosulfate copolymer, and mixtures thereof.

18. The conditioning detergent composition of claim 1 wherein the matrix soluble silicone is selected from the group consisting of trimethylsilylamodimethicone, dimethicone copolyol, amodimethicone and mixtures thereof.

19. The composition of claim 1 in the form of a shampoo, a conditioner, a body wash, a shower gel, or a bath.

20. A method for making a visually clear detergent composition substantially free of pearlizing agents, opacifiers and suspending agents, said method comprising the step of: adding an effective amount of a conditioning portion to a surfactant portion, wherein said conditioning portion comprises at least one cationic branched copolymer and at least one silicone which is different from said cationic branched polymer and is matrix soluble and wherein said surfactant portion comprises at least one of the following:
1. a nonionic surfactant;
2. an amphoteric surfactant; and
3. an anionic surfactant.
